(19) United States
(12) Patent Application Publication
(10) Pub. No.: US 2013/0053824 A1
(43) Pub. Date: Feb. 28, 2013

(54) Handheld Devices For Dilating Tissue

(71) Applicant: University of Cincinnati, Cincinnati, OH (US)

(72) Inventors: Allen Seiden, Cincinnati, OH (US); Mary Beth Privitera, Cincinnati, OH (US); Matthew Starkey, Cincinnati, OH (US); Ryan Lewis, Cincinnati, OH (US); Brigette K. Hofmann, Cincinnati, OH (US); Bradley A. King, Cincinnati, OH (US); Michael J. Sandmann, Cincinnati, OH (US)

(73) Assignee: University of Cincinnati, Cincinnati, OH (US)

(21) Appl. No.: 13/662,742

(22) Filed: Oct. 29, 2012

Related U.S. Application Data
(63) Continuation of application No. PCT/US2011/034540, filed on Apr. 11, 2011.

(51) Int. Cl. A61M 29/02; A61M 1/00

(52) U.S. Cl. 604/514; 604/96.01; 604/97.02; 604/99.01

(57) ABSTRACT

Embodiments of handheld devices for dilating tissue along the paranasal sinuses include a handle assembly that includes a grip portion, an inflation mechanism, and a digital actuation member configured to actuate the inflation mechanism. The handheld devices also include a rigid cannula that includes a distal end and a proximal end, where the rigid cannula is coupled to the handle assembly along the proximal end. The handheld devices further include an inflatable balloon coupled to an outside of the rigid cannula proximate to the distal end, an inflation lumen at least partially disposed along the rigid cannula and in fluid communication with the inflatable balloon and the inflation mechanism, and an aspiration lumen at least partially disposed along the rigid cannula and includes an aspiration port proximate to the distal end.

### CROSS REFERENCE TO RELATED APPLICATIONS

The present application is a continuation of International Application No. PCT/US2011/034540 filed Apr. 29, 2011, which claims the benefit of U.S. Provisional Application Ser. No. 61/329,833, titled “Manual Inflation of Balloon Using Triggers” filed Apr. 30, 2010.

### TECHNICAL FIELD

The present disclosure is generally directed to balloon catheters and, more specifically, balloon catheters attached to rigid cannulae.

### BACKGROUND

Inflammation of the sinus mucosa lining the sinus cavities causes sinusitis in patients. The mucosal lining includes mucosal glands and goblet cells that, together, secrete mucus. The mucus covers the mucosal lining of the sinuses and nose, and traps small particles such as dust, particulate matter, and allergens, as well as infectious agents such as bacteria, and prevents these particles from entering the respiratory system.

When the mucosal lining of the paranasal sinuses become inflamed, sinus ostia, or openings that connect sinuses to the naval cavity itself, tend to constrict or become obstructed. Constriction of the sinus ostia may lead to collection of mucus in the paranasal sinuses themselves, which may cause discomfort to the patient and lead to infection. Therapeutic treatment to relieve pressure in the paranasal sinuses may include dilation of the sinus ostia and drainage of the collected mucus. Accordingly, handheld devices that allow a medical practitioner to dilate tissue along the paranasal sinuses of a patient may be desired. Such a device may also facilitate atraumatic dissection when the need arises to surgically remove tissue and create larger openings.

### SUMMARY

In one embodiment, a handheld device for dilating tissue includes a handle assembly that includes a grip portion, an inflation mechanism, and a digital actuation member configured to actuate the inflation mechanism. The handheld device also includes a rigid cannula that includes a distal end and a proximal end, where the rigid cannula is coupled to the handle assembly along the proximal end. The handheld device further includes an inflatable balloon coupled to an outside of the rigid cannula proximate to the distal end, an inflation lumen at least partially disposed along the rigid cannula and in fluid communication with the inflatable balloon and the inflation mechanism, and an aspiration lumen at least partially disposed along the rigid cannula and includes an aspiration port proximate to the distal end.

In another embodiment, a handheld device for dilating tissue includes a handle assembly that includes a grip portion, an inflation mechanism, and a digital actuation member configured to actuate the inflation mechanism. The handheld device also includes a rigid cannula including a distal end and a proximal end, where the rigid cannula is coupled to the handle assembly along the proximal end, an inflatable balloon coupled to an outside of the rigid cannula proximate to the distal end, and an inflation lumen at least partially disposed along the rigid cannula and in fluid communication with the inflatable balloon and the inflation mechanism. The inflation mechanism includes a hydraulic system adapted to be operated by the digital actuation member, and translation of the digital actuation member displaces a working fluid from a working fluid reservoir to the inflatable balloon.

In yet another embodiment, a method of dilating tissue to facilitate atraumatic dissection within the nose and paranasal sinuses of a patient to thereby improve mucus drainage using a handheld device having a handle assembly, a rigid cannula coupled to the handle assembly, an inflatable balloon coupled to an outside of the rigid cannula, an aspiration lumen at least partially disposed along the inside of the rigid cannula, and an inflation mechanism in fluid communication with the inflatable balloon and operated by a digital actuation member including inserting the rigid cannula through a nasal opening of the patient as to place the inflatable balloon adjacent to the paranasal sinus of the patient. The method further includes depressing the digital actuation member such that the inflation mechanism causes the inflatable balloon to expand, and dilating tissue of the paranasal sinus.

### BRIEF DESCRIPTION OF THE DRAWINGS

The following detailed description of specific embodiments of the present disclosure can be best understood when read in conjunction with the drawings enclosed herewith.

FIG. 1 depicts a schematic representation of a handheld device for dilating tissue according to one or more embodiments shown and described herein;

FIG. 2 depicts a side partial sectional view of a handheld device for dilating tissue according to one or more embodiments shown and described herein;

FIG. 3 depicts a side sectional view of a handheld device for dilating tissue according to one or more embodiments shown and described herein;

FIG. 4A depicts a front sectional view of a handheld device for dilating tissue along line 4-4 of FIG. 2 according to one or more embodiments shown and described herein;

FIG. 4B depicts a front sectional view of a handheld device for dilating tissue along line 4-4 of FIG. 2 according to one or more embodiments shown and described herein;

FIG. 4C depicts a front sectional view of a handheld device for dilating tissue along line 4-4 of FIG. 2 according to one or more embodiments shown and described herein; and

FIG. 4D depicts a front sectional view of a handheld device for dilating tissue along line 4-4 of FIG. 2 according to one or more embodiments shown and described herein.

The embodiments set forth in the drawings are illustrative in nature and not intended to be limiting of the disclosure defined by the claims. Moreover, individual features of the drawings and disclosure will be more fully apparent and understood in view of the detailed description.

### DETAILED DESCRIPTION

The following text sets forth a broad description of numerous different embodiments of the present disclosure. The description is to be construed as exemplary only and does not describe every possible embodiment since describing every possible embodiment would be impractical, if not impossible, and it will be understood that any feature, characteristic, component, composition, ingredient, product, step or methodology described herein can be deleted, combined with or substituted for, in whole or part, any other feature, characteristic, component, composition, ingredient, product, step or methodology described herein. Numerous alternative embodiments could be implemented, using either current technology or technology developed after the filing date of this patent, which would still fall within the scope of the claims.

Embodiments of the present disclosure are directed to handheld devices that allow a medical practitioner to insert an inflatable balloon into an ostium along the paranasal sinuses of a patient, or along tissue planes leading to that ostium, and then to atraumatically dilate that ostium, or those tissue planes. Dilation of ostia may allow for evacuation of the paranasal sinuses from any mucus that has accumulated due to inflammation of the mucosal lining of the paranasal sinuses. Mucus retained in the paranasal sinuses of a patient may be evacuated using a suction device that is integrated into the handheld device, such that the medical practitioner using the handheld device can place the inflatable balloon in the ostia, dilate the ostia, and evacuate the paranasal sinus.

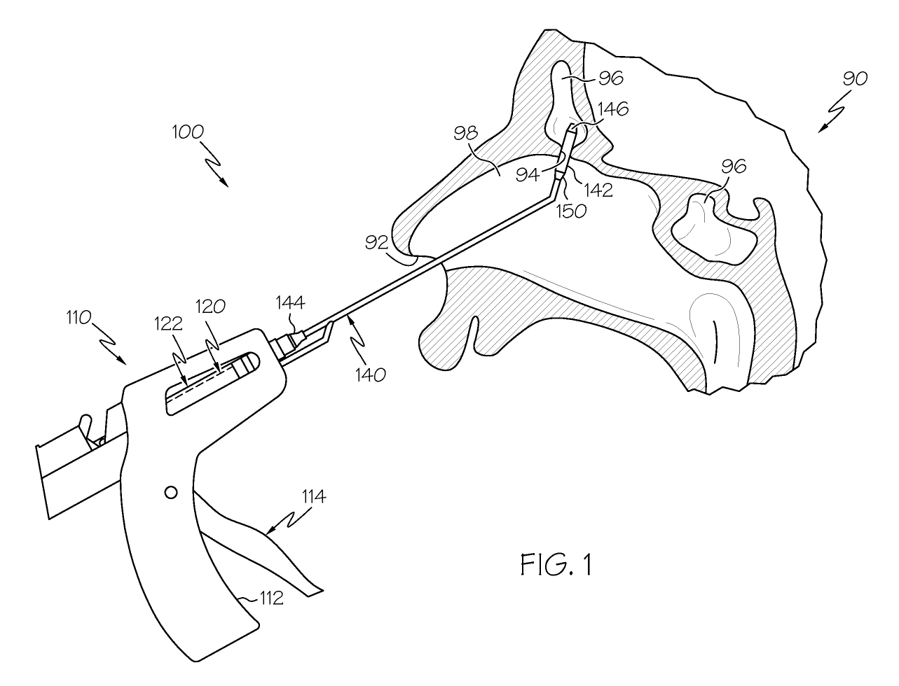

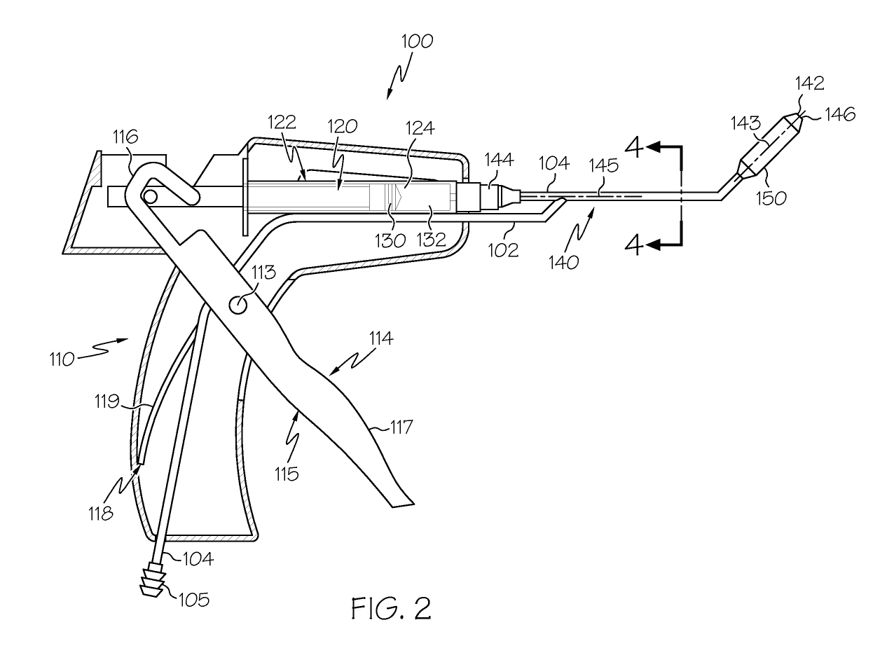

One embodiment of the handheld device 100 for dilating tissue is depicted in FIGS. 1 and 2. The handheld device 100 is depicted in a position relative to a patient 90. The handheld device 100 includes a handle assembly 110 and a rigid cannula 140 that extends from the handle assembly 110. As depicted, the rigid cannula 140 includes a proximal end 144 that attaches to the handle assembly 110 and a distal end 142. An inflatable balloon 150 is secured to the outside of the rigid cannula 140 in a location proximate to the distal end 142. The handle assembly 110 includes a grip portion 112, an inflation mechanism 120, and a digital actuation member 114. The digital actuation member 114 is configured to actuate the inflation mechanism 120.

Referring now to FIG. 2, components of the handheld device 100 according to one embodiment are shown in more detail. The inflation mechanism 120 may include a hydraulic system 122 that is operated by the digital actuation member 114. In the embodiment depicted in FIG. 2, the hydraulic system 122 includes a plunger 130 that is mechanically coupled to the digital actuation member 114. The plunger 130 forms a fluid-tight seal with a cylindrical tube 132. The plunger 130 and the cylindrical tube 132, together, form the working fluid reservoir 124, inside of which a working fluid may be stored for use in inflating the inflatable balloon 150. The cylindrical tube 132 is in fluid communication with the inflation lumen 104, such that translating the plunger 130 along the cylindrical tube 132 causes the working fluid to flow through the inflation lumen 104.

The handheld device 100 includes an inflation lumen 104 that is at least partially disposed along the rigid cannula 140. The inflation lumen 104 is in fluid communication with the inflatable balloon 150 and the inflation mechanism 120, such that when the inflation mechanism 120 pressurizes a working fluid, the working fluid travels along the inflation lumen 104 and into the inflatable balloon 150, placing the inflatable balloon 150 into an inflated state.

The digital actuation member 114 may include a trigger 115 that pivots about a fulcrum pin 113. As depicted in FIG. 2, the fulcrum pin 113 is statically coupled to the trigger 115, and the fulcrum pin 113 rotates about a bearing surface in the grip portion 112. However, the bearing surface may alternately be included within the trigger 115 and the fulcrum pin 113 statically coupled to the grip portion 112, such that the trigger 115 rotates about the fulcrum pin 113. The trigger coupling portion 116 is located along the trigger 115 opposite the fulcrum pin 113 from the trigger actuation portion 117. The trigger coupling portion 116 couples the trigger 115 to the inflation mechanism 120. Rotation of the trigger 115 about the fulcrum pin 113 is converted to translation of a portion of the plunger 130. As a medical practitioner applies a force to the trigger actuation portion 117 of the trigger 115, the trigger 115 rotates about the fulcrum pin 113 and the trigger coupling portion 116 applies a force that translates the plunger 130 forward along the cylindrical tube 132, thereby introducing working fluid into the inflation lumen 104.

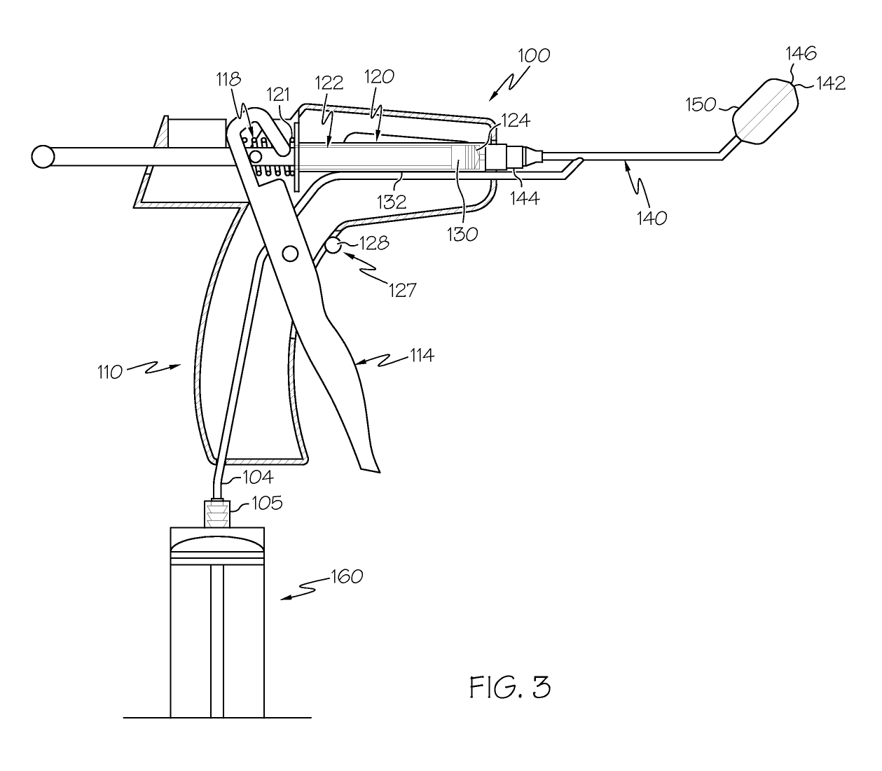

The handle assembly 110 may further include a return mechanism 118 that returns the digital actuation member 114 to its non-actuated position. As the digital actuation member 114 is coupled to the inflation mechanism 120, returning the digital actuation member 114 to its non-actuated position causes the inflation mechanism 120 to draw working fluid back into the working fluid reservoir 124, which displaces working fluid away from the inflatable balloon 150. In the embodiment depicted in FIG. 2, the return mechanism 118 is configured as a torsion spring 119 that is located in the grip portion 112 that applies a force that tends to rotate the digital actuation member 114 about the fulcrum pin 113 opposite the direction that a medical practitioner applies force to inflate the inflatable balloon 150. In another embodiment depicted in FIG. 3, the return mechanism 118 includes a spring, for example a compression spring 121, that applies a force to the plunger 130 in a direction that tends to increases the volume of the working fluid reservoir 124. In these embodiments, the return mechanisms 118 apply a force sufficient to draw working fluid down the inflation lumen 104 away from the inflatable balloon 150 as to deflate the inflatable balloon 150.

Referring again to FIG. 2, the handheld device 100 may also include an aspiration lumen 102 that is at least partially disposed along the rigid cannula 140. The aspiration lumen 102 extends to the distal end 142 of the rigid cannula 140. At the distal end 142 of the rigid cannula 140, the aspiration lumen 102 forms an aspiration port 146. At the opposite end of the aspiration lumen 102, an aspiration connector 105, for example a Luer Lock, is affixed to allow attachment of a suction mechanism (not shown).

Still referring to FIG. 2, the rigid cannula 140 defines a distal cannula axis 143 along the distal end 142 of the rigid cannula 140. The rigid cannula 140 also defines a proximal cannula axis 145 along the proximal end 144 of the rigid cannula 140. The distal end 142 of the rigid cannula 140 is inclined relative to the proximal end 144 of the rigid cannula 140, such that the distal cannula axis 143 is angled relative to the proximal cannula axis 145. In the embodiment depicted in FIG. 2, the distal cannula axis 143 is approximately 40 degrees from parallel with the proximal cannula axis 145, however the rigid cannula 140 may be formed such that the angle from parallel may be selected from the range from about 0 degrees to about 135 degrees. The angle from parallel of the distal cannula axis 143 to the proximal cannula axis 145 may be fixed for any particular rigid cannula 140. A medical practitioner may select from a plurality of rigid cannulae 140, each having a different angle from parallel of the distal cannula axis 143 to the proximal cannula axis 145. The selection of a rigid cannula 140 may be based on the anatomy of any particular patient 90.

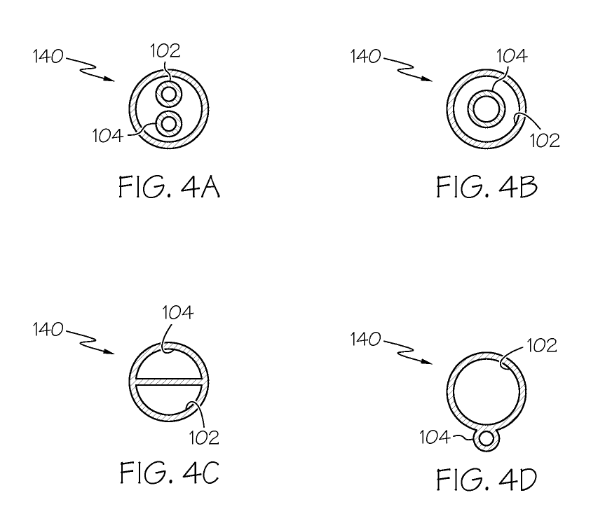

Referring now to FIG. 3, one embodiment of the handheld device 100 may include an inflation-limiting stop 127 that is adapted to hold the inflatable balloon 150 at maximum inflation without further intervention of the medical practitioner. As depicted in FIG. 4, the inflation-limiting stop 127 limits the rotation of the digital actuation member 114 when the digital actuation member 114 is in a fully depressed state (i.e., corresponding to a fully inflated state of the inflatable balloon 150). The inflation-limiting stop 127 engages the digital actuation member 114 to prevent additional working fluid from being introduced from the inflation mechanism 120 into the inflatable balloon 150. The inflation-limiting stop 127 also includes an inflation-limiting stop release 128. The medical practitioner using the handheld device 100 may disengage the inflation-limiting stop 127 from the digital actuation member 114 by applying a directional force to the inflation-limiting stop release 128. Other inflation-limiting stops that limit inflation of the inflatable balloon 150 by the inflation mechanism 120, for example, by limiting the relative motion of the plunger 130 along the cylindrical tube 132, are envisioned.

Cross sections of multiple embodiments of the rigid cannula 140 are shown in FIGS. 4A-4D. The embodiment depicted in FIG. 4A, the inflation lumen 104 and the aspiration lumen 102 are formed from discrete tubes that pass along the inside of the rigid cannula 140. In FIG. 4B, the inside of the rigid cannula 140 forms the inflation lumen 104, and the aspiration lumen 102 is disposed inside of a tube that is located coaxially with the rigid cannula 140. In FIG. 4C, both the inflation lumen 104 and the aspiration lumen 102 are formed within the rigid cannula 140. In FIG. 4D, the inflation lumen 104 is formed along the inside of the rigid cannula 140, while the aspiration lumen 102 is formed within a tube that is disposed along the outside of the rigid cannula 140. In this embodiment, the tube forming the aspiration lumen 102 may be permanently affixed to the rigid cannula 140. In each of these embodiments, the inflation lumen 104 is in fluid communication with the inflation mechanism 120 and the inflatable balloon 150, while the aspiration lumen 102 is in fluid communication with the aspiration connector 105 and the aspiration port 146. The inflation lumen 104 and the aspiration lumen 102 are fluidically isolated from one another, such that fluid that flows through the inflation lumen 104 does not flow through the aspiration lumen 102, and vice versa.

Referring again to FIG. 1, a medical practitioner operates the handheld device 100 by inserting the distal end 142 of the rigid cannula 140 through the nasal opening 92 of a patient 90. The medical practitioner may position the distal end 142 such that inflatable balloon 150, in a deflated state, is placed inside of one of the ostia 94 that connects the paranasal sinus 96 with the nasal canal 98, or within a tissue plane leading to that ostium. The medical practitioner may use an endoscope (not shown) inserted though the nasal opening 92 of the patient 90 to view the distal end 142 of the handheld device 100, such that the medical practitioner can visualize insertion of the inflatable balloon 150 within the ostium 94 or appropriate tissue plane to ensure accurate placement. Visualization techniques other than endoscopy may be used to verify accurate placement of the inflatable balloon 150 within the ostium 94.

When the medical practitioner is satisfied that the inflatable balloon 150 is accurately placed within the ostium 94, the medical practitioner may actuate the digital actuation member 114. As a medical practitioner applies a force that rotates the digital actuation member 114, the digital actuation member 114 causes the hydraulic system 122 to transfer a working fluid from a working fluid reservoir 124 along the inflation lumen 104 and into the inflatable balloon 150. Introduction of this working fluid places the inflatable balloon 150 in an inflated state. Because the digital actuation member 114 is configured to actuate the inflation mechanism 120, as the medical practitioner continues to depress the digital actuation member 114, the inflation mechanism 120 continues to inflate the inflatable balloon 150 located within the ostium 94 or tissue plane. The medical practitioner may continue to inflate the inflatable balloon 150 until such point that the desired inflation of the inflatable balloon 150, and corresponding dilation of the ostium 94, or tissue plane, has been achieved, or until the maximum inflation of the inflatable balloon 150 has been reached.

The rigid cannula 140 and the inflatable balloon 150 may be of a diameter that allows insertion of the inflatable balloon 150 within the ostium 94. The diameter of the rigid cannula 140 may be from about 2 mm to about 4 mm, and may be made from a material that is non-reactive with the tissue of a patient 90 and have a modulus of elasticity, or characteristic stiffness of the material, that is great enough such that the rigid cannula 140 does not perceptively flex while in operation. Materials satisfying these requirements include, but are not limited to, stainless steels, titanium, nickel, and alloys thereof. The rigidity of the rigid cannula 140 allows the medical practitioner to accurately and reliably place the distal end 142 of the rigid cannula 140 in a position relative to the ostium 94. Further, the rigidity of the rigid cannula 140 may allow the medical practitioner to apply a slight force to the tissues of the paranasal sinus 96 as to overcome any resistance encountered when inserting the inflatable balloon 150 into the ostium 94.

The inflatable balloon 150 may be made from a variety of materials that are resilient and are non-reactive with the tissue of a patient 90. In one embodiment, the inflatable balloon 150 may be made from polyethylene terephthalate (PET). The PET may be formed into a thin membrane-like surface that allows the inflatable balloon 150 to collapse onto the rigid cannula 140 when deflated. In the embodiment of the handheld device 100 depicted in FIG. 2, the inflatable balloon 150 may have an inflated outside diameter from about 3 mm to about 7 mm, and may have a length from about 16 mm to about 24 mm. Because an inflatable balloon 150 made of PET at this size is generally non-compliant (i.e., the wall of the inflatable balloon 150 does not stretch) the maximum diameter to which the inflatable balloon 150 may be inflated is controlled by the dimensions of the inflatable balloon 150 as manufactured. As such, the size of the inflatable balloon 150 as manufactured determines the maximum inflation of the inflatable balloon 150 within the paranasal sinuses of the patient 90. The inflatable balloon 150 itself may be attached to the outside of the rigid cannula 140 through a variety of permanent or temporary methods, including using adhesives or mechanical clamping systems. The inflatable balloon 150, the rigid cannula 140, and the attachment method of the inflatable balloon 150 to the rigid cannula 140 may be able to withstand pressures applied by the working fluid in excess of about 12 atmospheres.

The working fluid that is displaced by the inflation mechanism 120 along the inflation lumen 104 and into the inflatable balloon 150 may be an incompressible fluid. Because the fluid is incompressible, the force that a medical practitioner applies to the digital actuation member 114 will generally correspond to the pressure of the fluid in the inflatable balloon 150. In the embodiment depicted in FIGS. 1 and 2, the working fluid is saline. At the operating conditions of the handheld device 100, the saline remains incompressible throughout inflation and deflation of the inflatable balloon 150. Further, if there is a fluid leak along the inflation lumen 104 or the inflatable balloon 150, saline will not react with the tissue of the patient 90. Alternately, a compressible fluid, for example air, may be used as the working fluid.

The pressure of the working fluid inside the inflatable balloon 150, the inflation lumen 104, and the inflation mechanism 120 may provide a haptic feedback to the medical practitioner who is using the handheld device 100. The inflatable balloon 150 is inserted into the ostium 94 or appropriate tissue plane of the patient 90 in a deflated state. The medical practitioner applies a force to the digital actuation member 114 that causes the inflation mechanism 120 to displace working fluid along the inflation lumen 104 and into the inflatable balloon 150. As the inflatable balloon 150 contacts the tissue along the paranasal sinus 96, the ostium 94 or tissue plane may resist dilation and apply a resistive force to the inflatable balloon 150. The resistive force increases the pressure of the working fluid in the inflatable balloon 150. The medical practitioner may feel this increased pressure from contact with the digital actuation member 114. As the ostium 94 continues to dilate, an increased force is applied to the inflatable balloon 150 by the ostium 94 and the surrounding tissue of the paranasal sinus 96. The increased force increases the pressure of the working fluid within the inflatable balloon 150. The medical practitioner using the handheld device 100 may feel this increased pressure from contact with the digital actuation member 114. Thus, the digital actuation member 114 provides a haptic feedback to the medical practitioner that relates a pressure of the working fluid.

If the medical practitioner chooses to continue to fill the inflatable balloon 150 until it reaches its maximum inflation state, the inflation-limiting stop 127 will intervene to limit the amount of working fluid the inflation mechanism 120 may introduce to the inflatable balloon 150. The inflation-limiting stop 127 may mechanically prevent the medical practitioner from further introducing working fluid to the inflatable balloon 150. The medical practitioner using the handheld device 100 may feel the mechanical stop from contact with the digital actuation member 114. Thus, the digital actuation member 114 provides a haptic feedback to the medical practitioner that relates maximum inflation of the inflatable balloon 150. Further, in some embodiments, the inflation-limiting stop 127 may make an audible sound that the medical practitioner may perceive aurally.

With the ostium 94 dilated, the medical practitioner may aspirate the paranasal sinus 96 to remove accumulated mucus from the sinus cavity. With the inflatable balloon 150 still in an inflated state, the medical practitioner may apply suction to the aspiration lumen 102 by applying suction at the aspiration connector 105. As depicted in FIG. 3, a suction device 160 may be attached to the aspiration connector 105. The suction device 160 may apply a vacuum to the aspiration lumen 102 as to reduce the pressure of the aspiration lumen 102 in the region of the aspiration port 146. The reduced pressure about the aspiration port 146 draws fluid from the paranasal sinus 96 into the aspiration lumen 102 and towards the suction device 160. The suction device 160 may be manually operated by the medical practitioner or it may be machine-operated.

With the ostium 94 dilated to the desire of the medical practitioner, the pressure of the working fluid within the inflatable balloon 150 may be relieved by the medical practitioner releasing force on the digital actuation member 114. The return mechanism 118 draws the working fluid away from the inflatable balloon 150 and towards the working fluid reservoir 124. When the inflatable balloon 150 is fully deflated, the medical practitioner may remove the handheld device 100 from the nasal cavity of the patient 90, and free from the nasal opening 92.

It should now be understood that handheld devices for dilating tissues along the paranasal sinuses include a rigid cannula that allows a medical practitioner to accurately and reliably place an inflatable balloon within a constricted ostium or tissue plane. The handheld devices allow the medical practitioner to dilate the tissue, and receive direct feedback from the handheld device that relates the pressure inside the inflatable balloon and the stiffness of the tissue itself. The handheld device may further include an aspiration lumen that allows the medical practitioner to remove mucus from the paranasal sinus volume to provide relief to the patient.

It is further noted that terms like “preferably,” “generally,” “commonly,” and “typically” are not utilized herein to limit the scope of the claimed disclosure or to imply that certain features are critical, essential, or even important to the structure or function of the claimed disclosure. Rather, these terms are merely intended to highlight alternative or additional features that may or may not be utilized in a particular embodiment of the present disclosure.

For the purposes of describing and defining the present disclosure it is additionally noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

Having described the disclosure in detail and by reference to specific embodiments thereof, it will be apparent that modifications and variations are possible without departing from the scope of the disclosure defined in the appended claims. More specifically, although some aspects of the present disclosure are identified herein as preferred or particularly advantageous, it is contemplated that the present disclosure is not necessarily limited to these preferred aspects of the disclosure.

What is claimed is:

1. A handheld device for dilating tissue, comprising:
a handle assembly comprising a grip portion, an inflation mechanism, and a digital actuation member configured to actuate the inflation mechanism;
a rigid cannula comprising a distal end and a proximal end, wherein the rigid cannula is coupled to the handle assembly along the proximal end;
an inflatable balloon coupled to an outside of the rigid cannula proximate to the distal end;
an inflation lumen at least partially disposed along the rigid cannula and in fluid communication with the inflatable balloon and the inflation mechanism; and
an aspiration lumen at least partially disposed along the rigid cannula and comprising an aspiration port proximate to the distal end.

2. The handheld device of claim 1, wherein:
the inflation mechanism comprises a hydraulic system adapted to be operated by the digital actuation member; and
translation of the digital actuation member transfers a working fluid from a working fluid reservoir to the inflatable balloon through the inflation lumen.

3. The handheld device of claim 1, wherein:
the inflation mechanism comprises a plunger that forms a fluid-tight seal with a cylindrical tube;
the cylindrical tube is in fluid communication with the inflation lumen; and
the inflation mechanism is adapted to displace a working fluid along the inflation lumen and into the inflatable balloon as the plunger travels along the cylindrical tube.

4. The handheld device of claim 3, wherein as the inflatable balloon dilates tissue, the digital actuation member provides a haptic feedback that relates a pressure of the working fluid to a user.

5. The handheld device of claim 4, wherein a magnitude of the haptic feedback corresponds to a stiffness of the tissue being dilated.

6. The handheld device of claim 3, wherein the handle assembly further comprises a return mechanism adapted to displace the working fluid away from the inflatable balloon.

7. The handheld device of claim 6, wherein the return mechanism comprises a torsion spring, a compression spring, or a tension spring.

8. The handheld device of claim 1, wherein:
the digital actuation member comprises a trigger that pivots about a fulcrum pin; and
the trigger is coupled to the inflation mechanism.

9. The handheld device of claim 1, further comprising a suction device in fluid communication with the aspiration lumen.

10. The handheld device of claim 1, wherein the inflation mechanism comprises an inflation-limiting stop that is adapted to hold the inflatable balloon at maximum inflation without further intervention of a user.

11. The handheld device of claim 10, wherein the inflation mechanism further comprises an inflation-limiting stop release.

12. The handheld device of claim 10, wherein as the inflatable balloon approaches maximum inflation, the digital actuation member provides a haptic indication that relates engagement of the inflation-limiting stop to the user.

13. The handheld device of claim 1, wherein:
the rigid cannula defines a distal cannula axis along the distal end;
the rigid cannula defies a proximal cannula axis along the proximal end; and
the distal cannula axis is angled relative to the proximal cannula axis.

14. A handheld device for dilating tissue, comprising:
a handle assembly comprising a grip portion, an inflation mechanism, and a digital actuation member configured to actuate the inflation mechanism;
a rigid cannula comprising a distal end and a proximal end, wherein the rigid cannula is coupled to the handle assembly along the proximal end;
an inflatable balloon coupled to an outside of the rigid cannula proximate to the distal end; and
an inflation lumen at least partially disposed along the rigid cannula and in fluid communication with the inflatable balloon and the inflation mechanism,
wherein the inflation mechanism comprises a hydraulic system adapted to be operated by the digital actuation member, and translation of the digital actuation member transfers a working fluid from a working fluid reservoir to the inflatable balloon.

15. The handheld device of claim 14, wherein as the inflatable balloon dilates tissue, the digital actuation member provides a haptic feedback that relates a pressure of the working fluid to a user.

16. The handheld device of claim 14, the handle assembly further comprises a return mechanism adapted to displace the working fluid away from the inflatable balloon.

17. A method of dilating tissue to facilitate atraumatic dissection within the nose and paranasal sinuses of a patient to thereby improve mucus drainage using a handheld device having a handle assembly, a rigid cannula coupled to the handle assembly, an inflatable balloon coupled to an outside of the rigid cannula, an aspiration lumen at least partially disposed along the inside of the rigid cannula, and an inflation mechanism in fluid communication with the inflatable balloon and operated by a digital actuation member, the method comprising:
inserting the rigid cannula through a nasal opening of the patient as to place the inflatable balloon adjacent to the paranasal sinus of the patient;
depressing the digital actuation member such that the inflation mechanism causes the inflatable balloon to expand; and
dilating tissue of the paranasal sinus.

18. The method of dilating tissue of claim 17, further comprising applying suction to the aspiration lumen to aspirate fluid from the paranasal sinus.

19. The method of dilating tissue of claim 17, further comprising depressing the digital actuation member until a user experiences a desired haptic feedback provided by the digital actuation member, wherein a magnitude of the haptic feedback corresponds to a stiffness of the tissue being dilated.

20. The method of dilating tissue of claim 17, further comprising releasing the digital actuation member such that the inflation mechanism causes the inflatable balloon to contract.
